(19) United States
(12) Patent Application Publication
(10) Pub. No.: US 2016/0219812 A1
(43) Pub. Date: Aug. 4, 2016

(54) METHOD FOR PRODUCING HYBRID BRASSICA SEED

(71) Applicant: PIONEER HI-BRED INTERNATIONAL, INC., Johnston, IA (US)

(72) Inventors: CHRISTINA ELIZABETH MOLTZAHN, FORT MACLEOD, ALBERTA (CA); JOHN PAUL SCHMIDT, CHAMPAIGN, IL (US)

(21) Appl. No.: 14/906,064

(22) PCT Filed: Jul. 24, 2014
(86) PCT No.: PCT/US14/48007

§ 371 Date: Jan. 19, 2016

Related U.S. Application Data
(60) Provisional application No. 61/858,221, filed on Jul. 25, 2013.

(51) Int. Cl. A01H 1/02; A01H 5/10

(52) U.S. Cl. CPC A01H 1/02; A01H 5/10

(57) ABSTRACT

Method for producing hybrid Brassica seed comprising a ratio of four female rows to one male row. The rows are 14-30 inches in width. After pollination, the male rows are destroyed. This method increases the pollination rate of the female rows compared to current methods and reduces or eliminates the entanglement of female and male plants in the field.

### FIELD OF INVENTION

The invention is in the field of Brassica hybrid seed production. In particular, the invention relates to Brassica napus, and specifically canola, hybrid seed production.

### BACKGROUND

The present invention relates to a new method for producing hybrid Brassica seed. In particular, the invention relates to the production of Brassica napus hybrid seed, and more specifically, the invention relates to the production of canola hybrid seed.

Hybrid seed production is predominant in modern agriculture, and is one of the reasons for the rise in agricultural output in the past 50 years. Canola hybrid seed production is expanding greatly in Canada, Europe, the United States, as well as in other countries throughout the world.

Hybrids differ from conventional varieties in that they are produced by crossing two elite inbred lines (a male and female inbred) to produce hybrid seed. Hybrid seed has advantages over varietal seed or inbred seed due to heterosis. Elite inbred parental lines are chosen based on several criteria, including yield, combining ability, uniformity, disease resistance, insect resistance, oil and meal quality, drought and/or heat tolerance, reducing the time to crop maturity, abiotic stress tolerance, and better agronomic characteristics.

“Canola” refers to rapeseed (Brassica) which (1) has an erucic acid (C₂₂:1) content of at most 2 percent by weight based on the total fatty acid content of a seed, preferably at most 0.5 percent by weight and most preferably essentially 0 percent by weight; and (2) produces, after crushing, an air-dried meal containing less than 30 micromoles (μmol) glucosinolates per gram of defatted (oil-free) meal. Canola oil has the lowest level of saturated fatty acids of all vegetable oils. Canola is being recognized as an increasingly important oilseed crop and a source of meal in many parts of the world. The oil as removed from the seeds commonly contains a lesser concentration of endogenously formed saturated fatty acids than other vegetable oils and is well suited for use in the production of salad oil or other food products or in cooking or frying applications. The oil also finds utility in industrial applications. Additionally, the meal component of the seeds can be used as a nutritious protein concentrate for livestock.

Traditionally, canola hybrid seed is produced using a field management practice in which the female rows are grown in “bays.” Depending on the farmer's equipment, the bays of female plants are 18-22 feet wide with adjoining male rows that are 4-6 feet wide, alternating across the field.

The distance between rows can vary between 6-8 inches. After pollination, the male rows are destroyed using a mower (or flail chopper), leaving the female rows to develop seed, which are harvested, processed, and packaged for sale. One shortcoming of this approach is that the female rows farthest from the male rows (i.e. in the middle of the bay) have fewer pods per plant, thus smaller amounts of seed are produced from this area of the field. This could be a consequence of proximity to pollen, since producing hybrid canola seed relies on pollinators (e.g. honey bees and leaf cutter bees) to carry the male-plant pollen to the female plants.

Another problem with the bay management approach results when the female plants near the male plants become entangled with the male plants. This occurs because the distance between rows is small (i.e. 6-8 inches); consequently, some of the highest yielding female plants are abandoned in the field because of the risk of contamination of seed from the male plants.

### SUMMARY

The invention relates to the following:

<1> A method of producing Brassica hybrid seed, comprising;
(i) providing 3-8 rows of a female inbred line, wherein the female inbred line cannot self-pollinate;
(ii) providing 1-2 rows of a male inbred line adjacent to (i);
(iii) allowing pollination of the female inbred line to occur by the male inbred line;
(iv) after pollination, destroying the male inbred line; and
(v) after seed set, harvesting the hybrid seed from the female line.
   <2> The method of <1> wherein the rows are from about 14 to 30 inches apart.
   <3> The method of <1> wherein there are 4 rows of female inbred line.
   <4> The method of <1> wherein there is 1 row of male inbred line.
   <5> The method of <1> wherein the female inbred line is male sterile.
   <6> The method of <5> wherein the female inbred line is a cytoplasmic male sterile (cms) line or a genetic male sterile line.
   <7> The method of <6> wherein the female inbred line is an Ogura cytoplasmic male sterile (cms) line.
   <8> The method of <7> wherein the male inbred line comprises an Ogura fertility restorer gene, whereby the hybrid seed is fully restored.
   <9> The method of <1> wherein the female inbred line is self-incompatible (SI).
   <10> The method of <1> wherein the step of destroying the male inbred line comprises tilling, cutting, chopping or applying a herbicide.
   <11> The method of <10> wherein the herbicide is glyphosate, glufosinate, chlorsulfuron, or imidazolinone.
   <12> The method of <11> wherein the female inbred line is herbicide resistant.
   <13> The method of <12> wherein the female inbred line is glyphosate resistant, the male inbred line is not glyphosate resistant, and the herbicide that is applied to destroy the male inbred line is glyphosate.
   <14> The method of <10> wherein the male inbred line is destroyed by tilling.
   <15> The method of <10> wherein the male inbred line is destroyed by cutting.
   <16> The method of <1> wherein the rows are from about 18 to 25 inches apart.
   <17> The method of <1> wherein the rows are from about 20 to 22 inches apart.
   <18> The method of <1> wherein the step of allowing pollination to occur comprises the addition of insect pollinators.
   <19> The method of <1> wherein the Brassica is Brassica napus.
   <20> The method of <1> wherein Brassica is Brassica juncea.
   <21> The method of <1> wherein Brassica is canola quality Brassica napus.
   <22> A method of producing Brassica napus hybrid seed, comprising;
(i) providing 4 rows of a female inbred line, wherein the female inbred line is Ogura cytoplasmic male sterile;
(ii) providing 1 row of a male inbred line adjacent to (i), wherein the male inbred line comprises the Ogura fertility restorer gene;
(iii) allowing pollination of the female line to occur by the male inbred line;
(iv) after pollination, destroying the male inbred line; and
(v) after seed set, harvesting the hybrid seed from the female line, wherein the rows are about 20 inches apart.

### BRIEF DESCRIPTION OF THE FIGURES

FIG. 1 shows the method of hybrid seed production in the prior art (i.e. the “bay” system). Depending on the farmer's equipment, the bays of female plants are 18-22 feet wide with adjoining male rows that are 4-6 feet wide, alternating across the field. Dark grey represents the male inbred, light grey represents the female inbred.

FIG. 2 shows an embodiment of the hybrid seed production method of the present invention (the “row” system). Dark grey represents the male inbred, light grey represents the female inbred.

FIG. 3a Canola seed production using the bay system appears to have a greater incidence of Sclerotinia (light-colored plants) compared canola production using the row system (see FIG. 3b).

FIG. 3b Canola seed production using the row system.

FIG. 4 Canola seed production using the bay system (left) and the row system (right).

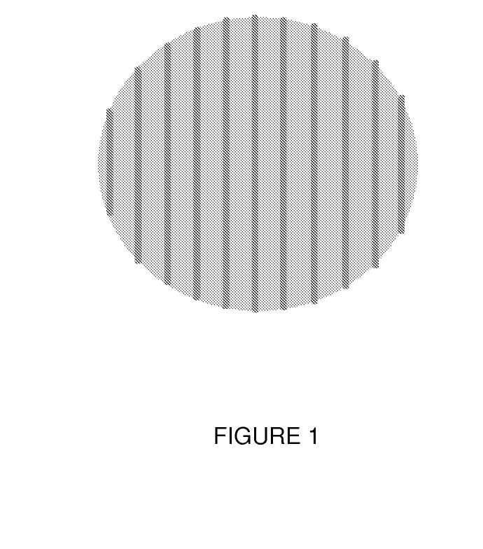

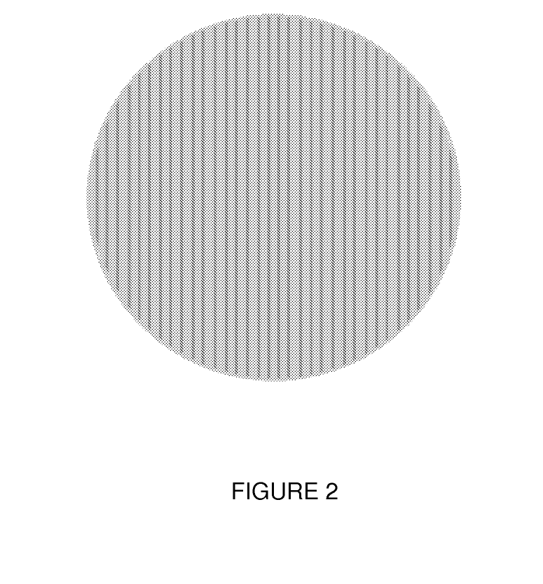

### DEFINITIONS

In the description which follows, a number of terms are used. In order to aid in a clear and consistent understanding of the specification, the following definitions and evaluation criteria are provided.

Female line: A female line is a population of male sterile plants that will accept pollen from another line (i.e. a pollen acceptor line). Accordingly, a female plant is a male sterile plant that will accept pollen from another plant.

Male line: A male line is a population that will donate pollen to another line (i.e. a pollen donor line). Accordingly, a male plant is a plant that donates pollen to a female plant.

Inbred line: An inbred line is a population that comprises nearly identical plants which are predominantly homozygous over a majority of their alleles. Inbred lines can be produced, for example, by repeated self-pollination or by double haploidy.

Hybrid seed: Hybrid seed is seed produced by crossing two different inbred lines (i.e. a female inbred line with a male inbred line). Hybrid seed is heterozygous over a majority of its alleles.

CMS: Abbreviation for cytoplasmic male sterility.

Erucic Acid Content: The percentage of the fatty acids in the form of C₂₂:1, as determined by one of the methods recommended by the WCC/RRC, being AOCS Official Method Ce 2-66 Preparation of Methyl esters of Long-Chain Fatty Acids or AOCS Official Method Ce 1-66 Fatty Acid Composition by Gas Chromatography.

Fatty Acid Content: The typical percentages by weight of fatty acids present in the endogenously formed oil of the mature whole dried seeds are determined. During such determination the seeds are crushed and are extracted as fatty acid methyl esters following reaction with methanol and sodium methoxide. Next the resulting ester is analyzed for fatty acid content by gas liquid chromatography using a capillary column which allows separation on the basis of the degree of unsaturation and fatty acid chain length. This procedure is described in the work of Daun, et al., (1983) J. Amer. Oil Chem. Soc. 60:1751 to 1754.

Glucosinolate Content. The total glucosinolates of seed at 8.5% moisture, as measured by AOCS Official Method AK-1-92 (determination of glucosinolates content in rapeseed-colza by HPLC), is expressed as micromoles per gram of defatted, oil-free meal. Capillary gas chromatography of the trimethylsilyl derivatives of extracted and purified desulfoglucosinolates with optimization to obtain optimum indole glucosinolate detection is described in “Procedures of the Western Canada Canola/Rapeseed Recommending Committee Incorporated for the Evaluation and Recommendation for Registration of Canola/Rapeseed Candidate Cultivars in Western Canada”. Also, glucosinolates could be analyzed using NIR (Near Infrared) spectroscopy as long as the instrument is calibrated according to the manufacturer's specifications.

Grain. Seed produced by the plant that is intended for food or feed use.

Herbicide Resistance: Resistance to various herbicides when applied at standard recommended application rates is expressed on a scale of 1 (resistant), 2 (tolerant), or 3 (susceptible).

Resistance/tolerance. The ability of a plant to withstand exposure to an insect, disease, herbicide, toxic substance, or other condition. A resistant/tolerant plant variety or hybrid will have a level of resistance/tolerance higher than a comparable wild-type variety or hybrid.

Row width or row spacing. Is a measurement from one row center (i.e. where the seed is placed) to the adjoining row center.

Destroying or damaging the male line. Upon application of a toxic substance (for example an herbicide) to which the male line is susceptible and the female line is resistant, the male plants will be damaged or destroyed whereas the female plants will be resistant. The degree of damage to the male line is such that any seed that is set on the male plants can be distinguished from the seed set on the female plants.

Seeds Per Pod. The average number of seeds per pod that is observed.

Seed Size. The weight in grams of 1,000 typical seeds is determined at maturity while such seeds exhibit a moisture content of approximately 5 to 6 percent by weight.

SI. Abbreviation for self-incompatible.

Seasonal Type. This refers to whether the line is considered to be primarily a Spring or Winter type of Brassica.

Bay system (bay planting system/bay production). The method of hybrid seed production in the prior art. Depending on the farmer's equipment, the bays of female plants are 18-22 feet wide with adjoining male rows that are 4-6 feet wide, alternating across the field. Row spacing is typically 6-8 inches.

Row system (row planting system/row production). The method of hybrid seed production of the present invention. The row system is typically four female rows adjoining one male row (4:1), wherein the rows are between 14-30 inches apart. Alternatively, the row system can be 3:1, 5:1, 6:1, 8:1 or 4:2, 5:2, 6:2, 7:2 or 8:2 female: male rows.

### DETAILED DESCRIPTION

The invention relates to a new method of hybrid seed production for Brassica species. Hybrid seed is produced by crossing two different lines (a male line (pollen donor) and a female line (pollen acceptor)) to produce F1 hybrid seed. The F1 hybrid seed will be heterozygous for all alleles (one allelic gene from each parent). In most cases, elite inbred lines are crossed to produce hybrid seed. The advantage of hybrid seed compared to varietal seed is due to heterosis.

In order to sell hybrid seed to growers (who in turn will plant the hybrid seed, produce a hybrid plant, collect the grain from the hybrid plant, and sell the grain to crushers for oil and meal production), it must be produced on a large scale, harvested, usually treated, and packaged in bags for commercialization. A commercial bag of hybrid seed must contain nearly 100% hybrid seed, i.e. there must be little or no contamination of inbred self-pollinated seed in the bag. There are two main reasons why there must be little or no contamination: (i) A grower pays a premium for hybrid seed and will not tolerate inferior inbred seed in his purchase and (ii) the presence of an inbred seed is an unintentional release of the breeders' elite inbred germplasm.

Further, the production of hybrid seed is a costly operation. When male and female plants are grown in a hybrid seed production field, only seed from the female plants is commercialized and sold. Therefore there is pressure to minimize male plants and maximize female plants. The fields must be carefully monitored for contaminants from weeds or other Brassica plants growing nearby which would contaminate the purity of the hybrid seed being produced.

Accordingly, the production of hybrid seed on a large scale for commercialization requires considerations of hybrid seed purity, increasing seed yield per unit area of land, and minimizing costs.

As stated above, the current field management practice for canola hybrid seed production utilizes female rows grown in bays of approximately 18-22 feet wide with adjoining male rows that are 4-6 feet wide, alternating across the field. The distance between rows is 6-8 inches. After pollination, the male rows are destroyed using a mower (or flail chopper), leaving the female rows to develop seed, which are harvested, processed, and packaged for sale. One shortcoming of this approach is that the female rows farthest from the male rows (i.e. in the middle of the bay) have fewer pods per plant thus smaller amounts of seed are produced from this area of the field. This could be a consequence of proximity to pollen, since producing hybrid canola seed on the female parent is dependent on pollinators (e.g. honey bees and leaf cutter bees) to carry the male-plant pollen to the female plants.

Another problem with the bay management approach results when the female plants near the male plants become entangled with the male plants. This occurs because the distance between rows is small (i.e. 7 inches); consequently, some of the highest yielding female plants are abandoned in the field because of the risk of contamination of seed from the male plants.

In contrast, the present invention provides a method of hybrid seed production comprising approximately 4 female inbred rows with one male inbred row, alternating across the field. A 4:1 scheme provides a similar proportion of female to male rows as the bay system. The proportion of female to male rows is important because when there are proportionally fewer female rows, production costs increase because there is less yield harvested per area of land leased from the grower. 3:1, 5:1, 6:1, 7:1 and 8:1 rows are also within the realm of the invention. Further 4:2, 5:2, 6:2, 7:2 and 8:2 rows are also within the realm of the invention.

Row spacing is typically 20-22 inches, but may vary between 14-30 inches. Any value between 14 and 30 is included in the scope of the invention. For example, 14, 15, 16, 17, 18, 19, 20, 21, 22, 23, 24, 25, 26, 27, 28, 29 or 30 inches can be used for row spacing. The value may also include fractions. The male row is used to provide pollen for the female rows. After pollination, the male row is destroyed (as is known to those skilled in the art, this can be done, for example, by tillage, chopping, mowing, use of herbicides (or other toxic substances) among other methods). The female rows are the only remaining plants in the field that develop mature seeds that are harvested, processed, and packaged for sale.

The 4:1 female:male row combination is a unique field management practice to produce hybrid canola seed. The 4:1 female:male row proportion results in closer proximity of female plants to male plants. This results in greater yield throughout the female rows (compared to bays), thus providing greater overall seed yield per unit area of land, resulting in greater return for the same input costs. Using the wider rows with the 4:1 configuration also eliminates (or minimizes) the problem of entangled female and male rows.

### Brassica Species and Seasonal Types

The present method of hybrid seed production can be used to produce hybrid seed for any Brassica species. For example, this method can be used to produce hybrid seed for Brassica napus, Brassica rapa, Brassica oleracea, Brassica nigra, Brassica carinata or Brassica juncea. In addition, this method can be used to produce both Brassica napus spring and Brassica napus winter types of hybrid seed.

### Pollination Control

Most Brassica species can self-pollinate as well as cross pollinate. For example, Brassica napus and Brassica juncea can self-pollinate and cross pollinate. Brassica rapa is mostly self-incompatible and therefore cannot self-pollinate readily, although self-compatible Brassica rapa named yellow sarsan is available in India. Brassica oleracea and Brassica nigra are mostly cross pollinated while Brassica carinata is self-pollinated. In order to facilitate hybrid seed production in species that can self-pollinate, pollination control systems can be used.

For example, Brassica napus canola plants, absent the use of sterility systems, are recognized to commonly be self-fertile with approximately 70 to 90 percent of the seed normally forming as the result of self-pollination. However, as stated above, self-pollination of the parental varieties can be controlled to make hybrid development more efficient.

On the most part, hybrid seed production requires inactivation of pollen produced by the female parent if the female parent can self-pollinate. Incomplete inactivation of the pollen provides the potential for self-pollination of the female plants. This inadvertently self-pollinated seed may be unintentionally harvested and packaged with hybrid seed. Similarly, because the male parent is grown next to the female parent in the field, there is also the potential that the male self-pollinated seed could be harvested and unintentionally packaged with the hybrid seed. Once the seed from the hybrid bag is planted, it is possible to identify and select these self-pollinated plants. These self-pollinated plants will be genetically equivalent to one of the inbred lines used to produce the hybrid. Though the possibility of inbreds being included in hybrid seed bags exists, the occurrence is rare because much care is taken to avoid such inclusions. In particular, self-pollinated seed from the male lines can be distinguished from the hybrid seed in the production facility. If the male line is damaged from the application of a toxic substance (for example, an herbicide) for which the male line is susceptible, the seed produced on the male line is shriveled and can be distinguished from hybrid seed. In addition, the self-pollinated plants can be identified and selected by one skilled in the art, through either visual or molecular methods.

The percentage of cross pollination may be further enhanced when populations of recognized insect pollinators at a given growing site are greater.

In developing improved new Brassica hybrid varieties, breeders may use self-incompatible (SI), cytoplasmic male sterile (CMS) or nuclear male sterile (NMS) Brassica plants as the female parent. In using these plants, breeders are attempting to improve the efficiency of seed production and the quality of the F₁ hybrids. If one of the parents is a SI, CMS or NMS plant that is incapable of producing pollen, only cross pollination will occur.

1. CMS System of Pollination Control

In one instance, production of F₁ hybrids includes crossing a CMS Brassica female parent with a pollen-producing male Brassica parent. To reproduce effectively, however, the male parent of the F₁ hybrid must have a fertility restorer gene (Rf gene). The presence of an Rf gene means that the F₁ generation will not be completely or partially sterile, so that either self-pollination or cross pollination may occur. Self-pollination of the F₁ generation to produce several subsequent generations is important to ensure that a desired trait is heritable and stable.

An example of a Brassica plant which is cytoplasmic male sterile and used for breeding is Ogura (OGU) cytoplasmic male sterile (Pellan-Delourme, et al., 1987) developed via protoplast fusion between radish (Raphanus sativus) and rapeseed (Brassica napus). A fertility restorer for Ogura cytoplasmic male sterile plants has been transferred from Raphanus sativus (radish) to Brassica by Instit. National de Recherche Agricole (INRA) in Rennes, France (Pelletier, et al., 1987). The OGU INRA restorer gene, Rf1 originating from radish, is described in WO 92/05251 and in Delourme, et al., (1991). Improved versions of this restorer have been developed. For example, see WO98/27806, oilseed brassica containing an improved fertility restorer gene for Ogura cytoplasmic male sterility.

Other sources and refinements of CMS sterility in canola include the Polima cytoplasmic male sterile plant, as well as those of U.S. Pat. No. 5,789,566, DNA sequence imparting cytoplasmic male sterility, mitochondrial genome, nuclear genome, mitochondria and plant containing said sequence and process for the preparation of hybrids; U.S. Pat. No. 5,973,233 Cytoplasmic male sterility system production canola hybrids; and WO97/02737 Cytoplasmic male sterility system producing canola hybrids; EP Patent Application Number 0 599042A Methods for introducing a fertility restorer gene and for producing F1 hybrids of Brassica plants thereby; U.S. Pat. No. 6,229,072 Cytoplasmic male sterility system production canola hybrids; U.S. Pat. No. 4,658,085 Hybridization using cytoplasmic male sterility, cytoplasmic herbicide tolerance, and herbicide tolerance from nuclear genes.

(i) Female development: As is known to those skilled in the art, the female line is developed by crossing a male sterile version of variety X (A-line) with a maintainer line of variety X (B-line). The A and B lines are genetically alike except the A-line carries the CMS cytoplasm, while B-line carries the normal cytoplasm.

(ii) Male development: As is known to those skilled in the art, a male parent or restorer (R line) of variety Y is developed. When crossed with the female parent, the R-line restores fertility to the resulting hybrid.

(iii) Hybrid development: A single cross hybrid is produced by crossing a female parent (male sterile inbred (A-line)×maintainer inbred (B-line)) by a restorer male (R-line), where the A and B lines are genetically alike except the A-line carries the CMS cytoplasm, while the B-line carries the normal B. napus cytoplasm.

2. Self-Incompatibility (SI) System of Pollination Control

Most Brassica rapa lines have a natural self-incompatibility system which do not allow them to self-pollinate. SI is commonly used in hybrid seed production in vegetable Brassicas (cabbages and cauliflower). The SI system has been suggested and tried in Brassica napus (see http://www.australianoilseeds.com/_data/assets/pdf_file/0004/6871/3_A_promisig_way_to_produce_B._napus_hybrid_seeds_by_self-incompatibility_{—}pollination_system.pdf, and citations therein, for example, Goring 1992, Gowers 1989, and Rahman 2005). In certain environments, SI inbred lines tend to self-pollinate resulting in lower hybridity in hybrid seed lots.

3. Genetic Male Sterility (GMS) System of Pollination Control

There are several methods of conferring genetic male sterility available, such as multiple mutant genes at separate locations within the genome that confer male sterility, as disclosed in U.S. Pat. Nos. 4,654,465 and 4,727,219 to Brar, et al., and chromosomal translocations as described by Patterson in U.S. Pat. Nos. 3,861,709 and 3,710,511. In addition to these methods, Albertsen, et al., U.S. Pat. No. 5,432,068, describe a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing, or turning “on”, the promoter, which in turn allows the gene that confers male fertility to be transcribed.

(A) Introduction of a deacetylase gene under the control of a tapetum-specific promoter and with the application of the chemical N-Ac-PPT (WO 01/29237).

(B) Introduction of various stamen-specific promoters (WO 92/13956, WO 92/13957).

(C) Introduction of the barnase and the barstar gene (Paul, et al., (1992) Plant Mol. Biol. 19:611-622).

For additional examples of nuclear male and female sterility systems and genes, see also, U.S. Pat. Nos. 5,859,341; 6,297,426; 5,478,369; 5,824,524; 5,850,014 and 6,265,640.

Also see, U.S. Pat. No. 5,426,041 (discovery relating to a method for the preparation of a seed of a plant comprising crossing a male sterile plant and a second plant which is male fertile), U.S. Pat. No. 6,013,859 (molecular methods of hybrid seed production) and U.S. Pat. No. 6,037,523 (use of male tissue-preferred regulatory region in mediating fertility).

### Pollination

Pollination can occur by natural insect pollinators or by providing additional pollinators in the field to encourage outcrossing. The additional insect pollinators may comprise honey bees (Apis mellifera) and leaf cutter bees (Megachile rotundata). As stated above, the ratio of 4:1 female:male rows allows most female plants to be sufficiently pollinated to produce a good yield of hybrid seed.

Destroying the Male Line after Pollination

After pollination has occurred, the male line can be destroyed by cutting, chopping, tilling, applying herbicides or other toxic substances, or by using other methods known to those skilled in art. Further combinations of the above methods of destroying the male line can be used. The male line is optimally destroyed after pollination and before seed set to avoid entanglement of seed pods with the female line. If a toxic substance is used, for example a herbicide, it is preferred that the female line is resistant to the herbicide while the male line is susceptible to the herbicide.

### Herbicide Application

A herbicide (or other toxic substance) can be used to destroy the male rows after pollination. Any herbicide can be used, for example, glyphosate, glufosinate ammonium, chlorsulfuron, imidazolinone, or other herbicides as is known to those skilled in the art.

When the male parent is susceptible to the herbicide and the female parent is resistant to the herbicide, there is no possibility that the herbicide will inadvertently kill some female plants. Accordingly, it is advantageous to use a herbicide resistant female parent. Examples of herbicide resistant Brassicas include: Roundup Ready (glyphosate) and Liberty Link (Liberty, Basta), varieties that were produced using genetic modification, and Clearfield (imidazolinone and chlorsulfuron resistant) varieties that were developed using mutagenesis. Accordingly, any one of these herbicide resistant Brassicas can be used as the female parent.

Herbicide resistant female lines can be produced by breeding, mutagenesis or genetic transformation. Examples of genes that confer resistance to a herbicide, include:

(A) A herbicide that inhibits the growing point or meristem, such as an imidazalinone or a sulfonylurea. Exemplary genes in this category code for mutant ALS and AHAS enzyme as described, for example, by Lee, et al., (1988) EMBO J. 7:1241, and Miki, et al., (1990) Theor. Appl. Genet. 80:449, respectively. See also, U.S. Pat. Nos. 5,605,011; 5,013,659; 5,141,870; 5,767,361; 5,731,180; 5,304,732; 4,761,373; 5,331,107; 5,928,937 and 5,378,824; and international publication WO 96/33270.

(B) Glyphosate (resistance imparted by mutant 5-enolpyruvl-3-phosphikimate synthase (EPSP) and aroA genes, respectively) and other phosphono compounds such as glufosinate (phosphinothricin acetyl transferase, PAT) and Streptomyces hygroscopicus phosphinothricin-acetyl transferase, bar, genes), and pyridinoxy or phenoxy propionic acids and cycloshexones (ACCase inhibitor-encoding genes). See, for example, U.S. Pat. No. 4,940,835 to Shah, et al., which discloses the nucleotide sequence of a form of EPSP which can confer glyphosate resistance. See also, U.S. Pat. No. 7,405,074, and related applications, which disclose compositions and means for providing glyphosate resistance. U.S. Pat. No. 5,627,061 to Barry, et al., also describes genes encoding EPSPS enzymes. See also, U.S. Pat. Nos. 6,566,587; 6,338,961; 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E; and 5,491,288; and international publications EP1173580; WO 01/66704; EP1173581 and EP1173582. A DNA molecule encoding a mutant aroA gene can be obtained under ATCC Accession Number 39256, and the nucleotide sequence of the mutant gene is disclosed in U.S. Pat. No. 4,769,061 to Comai. European Patent Application Number 0 333 033 to Kumada, et al., and U.S. Pat. No. 4,975,374 to Goodman, et al., disclose nucleotide sequences of glutamine synthetase genes which confer resistance to herbicides such as L-phosphinothricin. The nucleotide sequence of a phosphinothricin-acetyl-transferase gene is provided in European Application Number 0 242 246 to Leemans, et al., De Greef, et al., (1989) Bio/Technology 7:61, describe the production of transgenic plants that express chimeric bar genes coding for phosphinothricin acetyl transferase activity. See also, U.S. Pat. Nos. 5,969,213; 5,489,520; 5,550,318; 5,874,265; 5,919,675; 5,561,236; 5,648,477; 5,646,024; 6,177,616 B1 and 5,879,903. Exemplary of genes conferring resistance to phenoxy propionic acids and cycloshexones, such as sethoxydim and haloxyfop, are the Acc1-S1, Acc1-S2 and Acc1-S3 genes described by Marshall, et al., (1992) Theor. Appl. Genet. 83:435. See also, U.S. Pat. Nos. 5,188,642; 5,352,605; 5,530,196; 5,633,435; 5,717,084; 5,728,925; 5,804,425 and Canadian Patent Number 1,313,830.

(C) A herbicide that inhibits photosynthesis, such as a triazine (psbA and gs+ genes) and a benzonitrile (nitrilase gene). Przibilla, et al., (1991) Plant Cell 3:169, describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Numbers 53435, 67441 and 67442. Cloning and expression of DNA coding for a glutathione S-transferase is described by Hayes, et al., (1992) Biochem. J. 285:173.

(D) Acetohydroxy acid synthase, which has been found to make plants that express this enzyme resistant to multiple types of herbicides, has been introduced into a variety of plants (see, e.g., Hattori, et al., (1995) Mol Gen Genet 246:419). Other genes that confer tolerance to herbicides include: a gene encoding a chimeric protein of rat cytochrome P4507A1 and yeast NADPH-cytochrome P450 oxidoreductase (Shiota, et al., (1994) Plant Physiol 106:17), genes for glutathione reductase and superoxide dismutase (Aono, et al., (1995) Plant Cell Physiol 36:1687, and genes for various phosphotransferases (Datta, et al., (1992) Plant Mol Biol 20:619).

(E) Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1; and 5,767,373; and international publication WO 01/12825.

The female plants may carry two herbicide resistance genes for two different herbicides. For example, the male and female lines may be glyphosate resistant, and the female line may also carry a second herbicide resistance gene for a herbicide that would be used to eliminate the male plants. Some possible combinations are as follows:

| Female | Male | Application to kill males |
|---|---|---|
| Liberty + Glyphosate | Liberty | Glyphosate |
| Imidazolinone + Glyphosate | Imidazolinone | Glyphosate |
| Liberty + Glyphosate | Glyphosate | Liberty |
| Liberty + Imidazolinone | Imidazolinone | Liberty |
| Imidazolinone + | Glyphosate | Imidazolinone |
| Glyphosate | | |
| Imidazolinone + Liberty | Liberty | Imidazolinone |

The above table is provided as examples. Other herbicide combinations can also be used, as is known to those skilled in the art. In addition, the parents can have tolerance to two, three four, or more herbicide or other toxic substances.

Application of Toxic Substances, Other than Herbicides

Other toxic substances, for example, antibiotics, can be used.

### Harvesting the Hybrid Seed

After pollination of the female plants by the male plants, the female plants will set hybrid seed. The hybrid seed can be harvested by combine or by hand. Typically the plants are swathed first before combining. Alternatively, one can straight cut. If one straight cuts, a desiccant can be used, for example Reglone™.

### Screening of Harvested Seed for Quality Control

The harvested seed can be screened for small shriveled seed, weed seed, and other contaminants. For example, if the male plants have not been completely destroyed, but damaged, by the toxic substance, they may yield shriveled seed. This can be done, as is known to those skilled in the art, for example, by separating the seed with slotted screens, gravity table, and/or spiral equipment found at the seed processing facility. The small shriveled seed, weed seed, or other contaminants are discarded, leaving the clean hybrid seed as the commercial product.

### Seed Treatment and Packaging for Sale

“Treating a seed” or “applying a treatment to a seed” refers to the application of a composition to a seed as a coating or otherwise. The composition may be applied to the seed in a seed treatment at any time from harvesting of the seed to sowing of the seed. The composition may be applied using methods including but not limited to mixing in a container, mechanical application, tumbling, spraying, misting, and immersion. Thus, the composition may be applied as a slurry, a mist, or a soak. The composition to be used as a seed treatment can be a pesticide, fungicide, insecticide, or antimicrobial. For a general discussion of techniques used to apply fungicides to seeds, see “Seed Treatment,” 2d ed., (1986), edited by K. A Jeffs (chapter 9).

This novel method of hybrid seed production will be useful for producing new hybrid seed for commercialization. The new hybrid seed is a direct result of breeding elite inbred lines used as the parents in the hybrid seed production.

### Breeding Considerations

Female and male lines are specifically developed for hybrid seed production. In most cases, the female and male lines are inbred lines. There are numerous steps involving significant technical human intervention in the development of novel male and female lines. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single hybrid variety an improved combination of desirable traits from the parental germplasm. These important traits may include, but are not limited to higher seed yield, resistance to diseases and/or insects, tolerance to drought and/or heat, reducing the time to crop maturity, altered fatty acid profile(s), abiotic stress tolerance, improvements in compositional traits, and better agronomic characteristics.

These processes, which lead to the final step of marketing and distribution, can take from six to twelve years of significant technical human intervention starting from the time the first cross is made. Therefore, development of new canola hybrid varieties is a time-consuming process that requires precise forward planning, efficient use of resources, and a minimum of changes in direction. The development of a new hybrid variety typically involves the coordinated effort of a team of scientists, including plant breeders, molecular biologists, plant pathologists, entomologists, agronomists, biochemists, bioinformaticians, market analysts, and automation specialists.

Typically, female lines are developed that comprise a male sterility or pollination control system. Female lines may also be herbicide resistant.

Typically, male lines are developed that comprise a fertility restoration system. Male lines may also be herbicide resistant.

### Combining Ability

As is known to those skilled in the art, combining ability of a line, as well as the performance of the line per se, is a factor in the selection of improved canola lines that may be used as inbreds. Combining ability refers to a line's contribution as a parent when crossed with other lines to form hybrids. The hybrids formed for the purpose of selecting superior lines are designated test crosses. One way of measuring combining ability is by using breeding values. Breeding values are based on the overall mean of a number of test crosses. This mean is then adjusted to remove environmental effects and it is adjusted for known genetic relationships among the lines.

Canola breeding programs utilize techniques such as mass and recurrent selection, backcrossing, pedigree breeding and haploidy. For a general description of rapeseed and Canola breeding, see, Downey and Rakow, (1987) “Rapeseed and Mustard” In: Principles of Cultivar Development, Fehr, (ed.), pp 437-486; New York; Macmillan and Co.; Thompson, (1983) “Breeding winter oilseed rape Brassica napus”; Advances in Applied Biology 7:1-104; and Ward, et. al., (1985) Oilseed Rape, Farming Press Ltd., Wharfedale Road, Ipswich, Suffolk.

Molecular markers, including techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) and Single Nucleotide Polymorphisms (SNPs), may be used in plant breeding methods. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles in the plant's genome. The use of molecular markers in the selection process is often called Genetic Marker Enhanced Selection or Marker Assisted Selection (MAS).

### Production of Double Haploids

The production of double haploids can also be used for the development of plants with a homozygous phenotype in the breeding program. Methods for obtaining haploid plants are disclosed in P. V. Chuong & W. D. Beversdorf, High Frequency Embryogenesis through Isolated Microspore Culture in Brassica Napus L. and B. Carinata Braun, Plant Science 39, 219-226 (1985); Haploids in Crop Improvement II (C. E. Palmer, W. A. Keller & K. J. Kasha eds., Springer-Verlag 2005); L. S. Kott, Application of Double Haploid Technology in Breeding of Oilseed Brassica Napus, AgBiotech News Info 10(3), 69N-74N (1998); E. B. Swanson et al, Efficient Isolation of Microspore-Derived Embryos from Brassica Napus, Plant Cell Reports. 6, 94-97 (1987).

The production of doubled haploids can also be used for the development of inbreds in the breeding program.

### Transformation

The advent of new molecular biological techniques has allowed the isolation and characterization of genetic elements with specific functions, such as encoding specific protein products. Scientists in the field of plant biology developed a strong interest in engineering the genome of plants to contain and express foreign genetic elements, or additional, or modified versions of native or endogenous genetic elements in order to alter the traits of a plant in a specific manner. Any DNA sequences, whether from a different species, or from the same species that are inserted into the genome using transformation are referred to herein collectively as “transgenes”. Over the last fifteen to twenty years several methods for producing transgenic plants have been developed, and the present discovery, in particular embodiments, also relates to transformed versions of the inbred and or hybrid lines.

Numerous methods for plant transformation have been developed, including biological and physical plant transformation protocols. See, for example, Miki, et al., “Procedures for Introducing Foreign DNA into Plants” in Methods in Plant Molecular Biology and Biotechnology, Glick, and Genetic Transformation for the improvement of Canola World Conf, Biotechnol. Fats and Oils Ind. 43-46 (1988). In addition, expression vectors and in vitro culture methods for plant cell or tissue transformation and regeneration of plants are available. See, for example, Gruber, et al., “Vectors for Plant Transformation” in Methods in Plant Molecular Biology and Biotechnology, Glick and Thompson, Eds. (CRC Press, Inc., Boca Raton, 1993) pages 89-119. Any genes can be inserted into the Brassica species, including: (i) Genes that confer resistance to pests or disease; (ii) genes that confer herbicide resistance (as discussed above); (iii) genes that confer or contribute to an altered grain characteristic; (iv) genes that control pollination, hybrid seed production, or male-sterility; (v) genes that create a site for site specific DNA integration; (vi) genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress.

### INDUSTRIAL APPLICABILITY

The new method of hybrid seed production is useful for producing canola hybrid seed for commercial use. The hybrid seed is sown by growers and the resultant grain is harvested. The grain can be used for oil and/or meal production.

### EXAMPLES

### 1. Comparison of Row Planting System to Traditional Drilled Bay Plant System in Canola Seed Production Fields

### Summary

The primary objective of this study was to determine whether planting canola, for seed production, in 50-cm (20-in) rows in a 4:1 female:male row pattern improved seed yield compared to the traditional bay production. Bay production consists of drilling in 15-cm (6-in) rows with 6.1 m (20 ft) of female rows adjacent to 1.5 m (5 ft) of male rows. Side-by-side strips were planted in two and a half fields of canola seed production, including 17 total paired comparisons. Yield from row production was 27% greater than with bay production (2115 and 1660 kg ha⁻¹, respectively). Hybridity, based on Polymerase Chain Reaction (PCR), was high for both systems (93%) and Thousand Seed Weight was the same (4.9 g 1000⁻¹). This is the second year of results, and row production provides an attractive alternative to bay production for increasing yield in canola seed production.

### Background Information

Drills have traditionally been used for planting canola for hybrid seed production, alternating 6.1 m (20 ft) of female rows with 1.5 m (5 ft) of male rows. Row spacing with drills is usually about 15 cm (6 in). Seed growers have the equipment and skills to produce canola seed with this approach, generally known as bay production. However, one of the perceived shortcomings of this approach is that yield in the middle of the female bay is often less than the yield at the edge of the bay (i.e. near the male bay). This yield reduction is anecdotally attributed to a lower transfer of pollen from the male plants to female plants because of: 1) simply greater distance for bees to travel between male and female plants or 2) distance and a more closed canopy from the narrow row spacing (i.e. a longer and more tortuous path for the bee). Wider row spacing and a shorter distance from male plants to female plants may provide an opportunity to improve overall canola seed yield. Using a 50-cm (20-in) row spacing and a female:male row ratio of 4:1 would result in the same female to male proportion in a field, open the inter-row canopy, and decrease the farthest travel distance for bees between female and male plants.

### Results and Conclusions

Seed yield from row production was 455 kg ha⁻¹ (407 lb acre⁻¹) greater, or 27% more, than yield from bays (Table 1).

In addition to the effects on yield, row production might also result in better management of off-types, because wider row spacing may result in easier identification of off-types. However, hybridity, based on PCR test results, was not different between the two treatments; both had about 93% hybridity (Table 1).

TABLE 1
Canola seed yield, PCR hybridity, and Thousand Seed Weight for
Row and Bay seed production.

| | Yield^{†} | PCR Hybridity‡ | TSW^{§} |
|---|---|---|---|
| Treatment | kg (total ha)⁻¹ | % | g 1000⁻¹ |
| Row | 2115a | 92.6 | 4.9 |
| Bay | 1660b | 93.1 | 4.9 |

^{†}Means followed by different letter were significantly different
‡PCR = polymerase chain reaction
^{§}TSW—thousand seed weight

Thousand Seed Weight (TSW) was also the same for both treatments (4.9 g (1000 seed)⁻¹). An additional impact that rows might have on canola seed production is the variability in seed size. Although variability in TSW between rows and bays was not statistically different, there was a consistent trend in increasing uniformity with rows (P=0.1; Table 2). Row production appeared to provide more uniform seed size than bay production.

TABLE 2
Standard deviation for Thousand Seed Weight for each field.

Standard Deviation

| | |
|---|---|
| Bay | Row |

| | |
|---|---|
| Field | g (1000 seeds)⁻¹ |

| | | |
|---|---|---|
| 1043-11 | 0.18 | 0.14 |
| 1067-09 | 0.33 | 0.18 |
| 1067-11 | 0.26 | 0.21 |

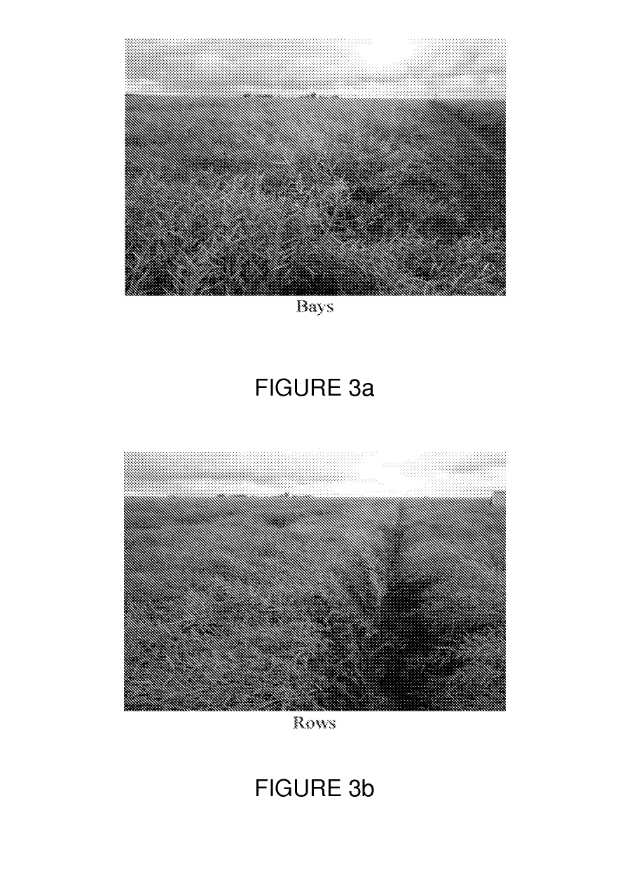

Although not quantified, the incidence of Sclerotinia appeared to be greater in the bays than in the rows (FIG. 3). A more closed canopy with a bay production system is more likely to contribute to Sclerotinia than the extra canopy space provided with a row production system.

### Study Description

Two and a half canola seed production fields (55, 52, and 26 ha or 135, 128, and 64 acres) near Lethbridge, AB, Canada were selected for this study. Inbreds grown were for hybrid 45H31, but inbreds for other hybrids can be used, as is known to those skilled in the art. Each whole field was divided into 14 strips and the half field into six strips. Each strip was 55-m (180-ft) wide. Bays and rows were assigned to alternating strips across the field and adjoining strips of each treatment were paired to be analyzed as paired t-tests.

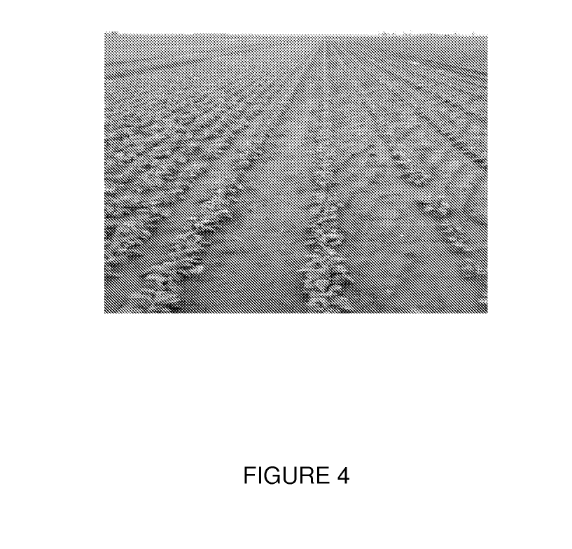

Each bay treatment system consisted of 6.1 m (20 ft) of female rows adjacent to 1.5 m (5 ft) of male rows. Row spacing for the bays was 15 cm (6 in) (FIG. 4). The row treatment system consisted of alternating four female rows with one male row (4:1). Row spacing for the row treatment was 50 cm (20 in). The area of each strip was determined with a global position system (GPS). Each strip was swathed and then combined with either a CASE IH 2388 or 2188 for seed yield determination. Seed from each individual strip was hauled in a grain truck to a scale to determine seed weight. A sample was collected periodically from the clean grain bin of the combine from each strip to determine Thousand Seed Weight (TSW), Green Seed, and for hybridity, based on Polymerase Chain Reaction analysis.

Data were analyzed using a paired t-test in SAS (SAS Institute Inc. 2011. SAS/STAT® 9.3 User's Guide. Cary, N.C.: SAS Institute Inc.). While treatments were not randomly assigned to each adjoining paired strips, a paired t-test analyses depends on randomization or uniformity in experimental units. Any trend or bias across the field was evaluated two different ways: 1) a regression of yield as a function of distance and 2) by switching the assignment of pairs in the paired t-test to the other adjoining strip. The yield comparison between treatments was affected by less than 5%, thus assuming uniformity in experimental units appeared to be reasonable for the paired t-test analysis. Any across-the-field trend or bias effect on TSW, Green Seed, and hybridity was assumed to be negligible. Statistical differences were considered significant at P<=0.05.

### Conclusion

The results described above are results from a second year comparing row to bay production systems. These results corroborate results from the first year experiment, when the study was conducted in two fields and there was a 22% yield increase with row production compared to bay production. A 4:1 female:male row combination increased yield 25% compared to using the traditional bay approach.

The foregoing invention has been described in detail by way of illustration and example for purposes of exemplification. However, it will be apparent that changes and modifications are considered to be within the scope of the present invention.

### Example 2

### Comparison of 8:2 Female:Male Row Pattern Planting System to Traditional Drilled Bay Plant System in Canola Seed Production Fields

The primary objective of this study was to determine whether planting canola, for seed production, in 50-cm (20-in) rows in a 8:2 female:male row pattern improved seed yield compared to the traditional bay production. Bay production consists of drilling in 15-cm (6-in) rows with 6.1 m (20 ft) of female rows adjacent to 1.5 m (5 ft) of male rows. Side-by-side strips were planted in one field of canola seed production near Enchant Alberta, including 4 paired comparisons. Yield from row production was 18% greater than with bay production (2485 and 2108 kg ha-1, respectively).

Seed yield from row production was 377 kg ha-1 (337 lb acre-1) greater, or 18% more, than yield from bays. Thousand Seed Weight (TSW) was also the same for both treatments (4.5 g (1000 seed)-1).

### Example 3

### Ration of 9:3 Female:Male Row Pattern Planting System in Canola Seed Production Fields

The primary objective of this study was to determine whether increasing proximity to male, for seed production, in 56-cm (22-in) rows improved seed yield. The two treatments included the 10:3 female:male adjacent to 8:3 female:male which were planted in side-by-side strips in four fields of canola seed production near Enchant Alberta. Multiple comparisons of plants will be made within each field. An advantageous difference is expected compared with traditional planting methods.

1. A method of producing Brassica hybrid seed, comprising;
(i) providing 3-8 rows of a female inbred line, wherein the female inbred line cannot self-pollinate;
(ii) providing 1-2 rows of a male inbred line adjacent to (i);
(iii) allowing pollination of the female inbred line to occur by the male inbred line;
(iv) after pollination, destroying the male inbred line; and
(v) after seed set, harvesting the hybrid seed from the female line

2. The method of claim 1, wherein the rows are from about 14 to about 30 inches apart.

3. The method of claim 1, wherein there are 4 rows of female inbred line in step (i).

4. The method of claim 3, wherein there is 1 row of male inbred line in step (ii).

5. The method of claim 1, wherein the female inbred line is male sterile.

6. (canceled)

7. The method of claim 5, wherein the female inbred line is an Ogura cytoplasmic male sterile (cms) line.

8. The method of claim 7, wherein the male inbred line comprises an Ogura fertility restorer gene, whereby the hybrid seed is fully restored.

9. The method of claim 1, wherein the female inbred line is self-incompatible (SI).

10. The method of claim 1, wherein the step of destroying the male inbred line comprises tilling, cutting, chopping or applying a herbicide.

11. The method of claim 10, wherein the male inbred line is destroyed by a herbicide selected from glyphosate, glufosinate, chlorsulfuron, or imidazolinone.

12. The method of claim 1, wherein the female inbred line is herbicide resistant.

13. The method of claim 1, wherein the female inbred line is glyphosate resistant, the male inbred line is not glyphosate resistant, and the herbicide that is applied to destroy the male inbred line is glyphosate.

14. The method of claim 10, wherein the male inbred line is destroyed by tilling or chopping.

15. (canceled)

16. The method of claim 1, wherein the rows are between 18-25 inches apart.

17. The method of claim 16, wherein the rows are about 20-22 inches apart.

18. The method of claim 1, wherein the step of allowing pollination to occur comprises the addition of insect pollinators.

19. The method of claim 1, wherein the Brassica is Brassica napus.

20. The method of claim 1, wherein the Brassica is Brassica juncea.

21. The method of claim 19, wherein the Brassica is canola quality Brassica napus.

22. A method of producing Brassica napus hybrid seed, comprising;
(i) providing 4 rows of a female inbred line, wherein the female inbred line is Ogura cytoplasmic male sterile;
(ii) providing 1 row of a male inbred line adjacent to (i), wherein the male inbred line comprises the Ogura fertility restorer gene;
(iii) allowing pollination of the female line to occur by the male inbred line;
(iv) after pollination, destroying the male inbred line; and
(v) after seed set, harvesting the hybrid seed from the female line, wherein the rows are about 20 inches apart.
